(19) United States
(12) Patent Application Publication
(10) Pub. No.: US 2026/0248550 A1
(43) Pub. Date: Aug. 27, 2026

(54) RESECTOSCOPE DEVICE AND RESECTOSCOPE

(71) Applicant: KARL STORZ SE & Co. KG, Tuttlingen (DE)

(72) Inventors: Peter SCHWARZ, Tuttlingen (DE); Christian GRAF, Tuttlingen (DE)

(73) Assignee: KARL STORZ SE & Co. KG, Tuttlingen (DE)

(21) Appl. No.: 19/546,021

(22) Filed: Feb. 20, 2026

(30) Foreign Application Priority Data
Feb. 24, 2025 (DE) 102025106810.0

(51) Int. Cl. A61B 18/14; A61B 1/00; A61B 1/015; A61B 1/05; A61B 1/307; A61B 18/00

(52) U.S. Cl. CPC A61B 18/14; A61B 1/00066; A61B 1/00135; A61B 1/00193; A61B 1/015; A61B 1/05; A61B 1/307; A61B 2018/00202; A61B 2018/00517; A61B 2018/00601; A61B 2018/0091; A61B 2018/00982; A61B 2018/1407

(57) ABSTRACT

The present disclosure relates to a resectoscope device comprising an insertion sleeve that can be fed into a cavity of a patient and has an oval cross-section, and a shaft that extends through the insertion sleeve and has a first shaft portion and a second shaft portion arranged in different positions along a longitudinal axis of the shaft, wherein, at least in an application configuration, the first shaft portion is arranged in the insertion sleeve and is rotatable within the insertion sleeve, and wherein the second shaft portion in the application configuration is arranged outside the insertion sleeve.

### CROSS-REFERENCE TO RELATED APPLICATIONS

This application claims priority of German Patent Application No. DE 102025106810.0 on Feb. 24, 2025, the contents of which are incorporated herein.

The present disclosure relates to a resectoscope device and a resectoscope.

A resection is the surgical removal of tissue from an organ or a tumor. Depending upon the objective and anatomical circumstances, different types of resections can be performed. In cases of difficult-to-access tissue, surgical removal can be performed endoscopically using a resectoscope. Such a resectoscope is used, for example, in transurethral resection, in which diseased tissue is removed from the bladder or prostate. The operation is performed endoscopically through the urethra of the patient, without the need for an incision.

A conventional resectoscope for transurethral resection comprises a shaft, at the distal end portion of which an observation optic, a rinsing device, and a resection tool are arranged. The shaft allows access to the bladder through the urethra and provides stability during the procedure. The distal end portion of the shaft can therefore be positioned during the procedure by sliding the shaft back and forth along the urethra and by pivoting the shaft in the bladder. A proximal portion of the shaft is coupled to an operating module that is held and moved by a user during the procedure. The operating module also comprises an actuating device by means of which the resection tool can be moved relative to the distal end portion.

A resection using such a conventional resectoscope is usually performed as follows. The user grasps the resectoscope at the operating module. He then pushes the shaft through the urethra of the patient in such a way that the distal end portion of the shaft protrudes into the bladder. The bladder is then filled using the rinsing device. Now the imaging device is activated, and the user gets an overview of the inside of the bladder through endoscopic observation. In this way, he can identify the tissue sites to be removed within the bladder. To remove them, the user pushes the shaft forwards and pivots it as needed until the distal end portion is near the affected tissue site. He then adjusts the imaging device so that the tissue site is clearly visible in the field of view. To ablate the tissue, the user now operates the actuating device, thereby pushing the resection tool back and forth. In an iterative process, the tissue can be ablated layer by layer by actuating the actuating device. To reach a larger region of tissue with the resection tool, which is regularly necessary, the user additionally moves the shaft along the urethra.

In the manner described, transurethral resection can be used primarily for the treatment of superficial bladder cancer by resection in a urinary bladder. The inventors of the present disclosure have recognized that this is difficult for the user to perform and that complications may arise. During the resection, the user must, according to the prior art, strongly pivot the resectoscope and move it intensively along the urethra. This can impair the precision of tissue ablation. Furthermore, complications can occur for the patient, with the back-and-forth movement of the shaft in particular often leading to postoperative discomfort for the patient. For example, injuries to muscles and tissue that are not directly visible or lead to severe irritation of the mucous membranes can occur during the procedure. These complications can subsequently lead to urinary incontinence. As mentioned, however, it is regularly absolutely necessary to push the shaft back and forth in the urethra in order to complete the treatment.

Furthermore, the inventors recognized that the use of an insertion sleeve can reduce friction between the shaft and the urethra. Similarly to a trocar, the insertion sleeve can provide access to the bladder, but can be a component of the resectoscope device. In this case, the shaft itself substantially no longer comes into direct contact with the urethra, but extends within the insertion sleeve.

However, the inventors have found that the overall diameter of the insertion sleeve and shaft is larger than the diameter of a conventional shaft that directly contacts the urethra, as is the case with a conventional resectoscope. The larger diameter can lead to greater tissue stretching, which, compared to using a resectoscope without an insertion sleeve, can result in increased tissue stress and potential injuries.

Proceeding from the prior art, the object addressed by the disclosure is that of providing a resectoscope by means of which can be carried out gently for a patient.

The object is achieved according to the disclosure by a resectoscope device and a resectoscope as are described herein and defined in the claims.

The present disclosure provides for the provision of a resectoscope device. Said device comprises an insertion sleeve that can be fed into a cavity of a patient and has an oval cross-section, and a shaft that extends through the insertion sleeve and has a first shaft portion and a second shaft portion that are arranged in different positions along a longitudinal axis of the shaft. At least in an application configuration, the first shaft portion is arranged in the insertion sleeve and is rotatable within the insertion sleeve. In the application configuration, the second shaft portion is located outside the insertion sleeve.

The present disclosure also provides for the provision of a resectoscope having a resectoscope device according to the disclosure.

The features according to the disclosure enable gentle resection for the patient. The installation space available is used efficiently, which can reduce the overall size compared to an insertion sleeve with a circular cross-section. Furthermore, an insertion sleeve with a cross-sectional geometry that is favorably adapted to anatomical conditions can be provided, thus avoiding unnecessary trauma. The features according to the disclosure minimize tissue stretching, which further reduces the risk of injury.

The inventors also noted that a shaft of a resectoscope device typically has different functional units along its length, each requiring a different amount of installation space. In the case of an insertion sleeve with a circular cross-section, the radius is always determined by the portion requiring the most space. However, this large radius is not required along the longitudinal extension of the shaft, since some shaft portions require only a smaller radius.

By using an insertion sleeve with an oval cross-section, the installation space can be used more efficiently. In particular, along the portions of the shaft that require little installation space, such as the first shaft portion, an unnecessarily large radius of the cross-section of the insertion sleeve is avoided.

In addition, the inventors recognized that the oval cross-section allows certain functional units to be formed by both the insertion sleeve and the shaft together. In other words, the insertion sleeve can perform additional functions beyond reducing friction. These additional functions are achieved through the specific shape of the cross-section, allowing for more efficient use of the installation space and a less complex resectoscope device. The shaft shape and the insertion sleeve shape can thus work together specifically to achieve certain functionalities.

A “resectoscope” can be understood as a medical instrument for the endoscopic removal of tissue within a hollow organ, in particular the bladder and/or the prostate. A resectoscope can enable minimally invasive procedures, especially through controlled resection movements and/or endoscopic imaging.

The term “resectoscope device” is to be understood as meaning, in particular, a preferably functional component, in particular a subassembly and/or a structural and/or functional component of a resectoscope. Preferably, the resectoscope device can form the resectoscope at least partially, preferably at least to a large extent, and particularly preferably completely. For example, the resectoscope device can be configured to be inserted at least partially and preferably at least to a large extent into a cavity or into, in particular, an artificial and/or natural cavity, in particular a body cavity, in particular to inspect it and to modify parts thereof, in particular to remove it. The resectoscope device can be a medical resectoscope device.

In the context of this disclosure, “configured” can be understood to mean, in particular, programmed, formed, designed, and/or equipped. The fact that a component is configured for a specific function can be understood within the context of this disclosure in particular to mean that the component fulfills and/or executes this specific function in at least one application and/or operating state.

The insertion sleeve can be designed as an elongated tube and/or comprise, in particular, such a tube along whose longitudinal axis a guide channel can run. In particular, it can be configured to provide access to a bladder of a patient. It can be positioned in and/or inserted into the urethra of the patient. In this arrangement, the longitudinal axis and, in particular, the guide channel can extend along the urethra. In this case, the insertion sleeve extends from a proximal side to a distal side. The shaft and/or the resection tool can be guided at least partially and/or portion by portion in the guide channel and, in particular, can be advanced in a guided manner.

In some embodiments, the shaft may be guided in the insertion shaft. In particular, the shaft can be guided linearly, in particular coaxially with the insertion sleeve, with its longitudinal axis along a longitudinal axis of the insertion sleeve. The insertion sleeve can therefore accommodate the shaft, in particular on a portion that can be arranged within the urethra. Similarly, other assemblies may be accommodated in the insertion sleeve-for example, a resection tool with a tool tip used to remove tissue and/or a rinsing device with a supply channel and a suction channel. The insertion sleeve can be captively attached to the shaft. In other words, the insertion sleeve can be movable relative to the shaft and attached to the shaft. Intuitive operation can be achieved in particular if the shaft, in particular the first shaft portion, has a linear degree of freedom of movement and a rotational degree of freedom of movement relative to the insertion sleeve-to be specific, can move axially and can rotate, but is stationary with respect to a radial position. In some embodiments, a rotational position can be fixed and/or definable-for example, by the shape of the second shaft portion and/or a projection on the first shaft portion. The resectoscope device may comprise a user-operable locking mechanism for this purpose, by means of which an axial position can be defined. Particularly intuitive operation is achieved if the shaft, in particular the first shaft portion, is linearly movable and rotatable relative to the insertion sleeve.

In other words, the insertion sleeve can function like a trocar, specifically a captive trocar, as known from minimally invasive surgery. It can therefore provide trocar-like access to the cavity, in particular the bladder.

The insertion sleeve can have a longitudinal axis along which the shaft is movable and/or extends. The longitudinal axis can extend in parallel with, in particular coaxially with, a main extension direction of the insertion sleeve. When the insertion sleeve is at least partially fed into the cavity, the longitudinal axis of the shaft may also extend in parallel with the main extension direction of the insertion sleeve and/or the longitudinal axis thereof.

The insertion sleeve may have a smaller longitudinal extension than the shaft, the first shaft portion and/or the second shaft portion. In some embodiments, the longitudinal extension of the insertion sleeve can, for example, be at most 95%, in particular at most 80%, preferably at most 70%, and particularly preferably at most 50%, of the longitudinal extension of the shaft, the first shaft portion, and/or the second shaft portion. The longitudinal extension can mean the extension along the longitudinal axis. The shaft can, for example, be between 20 cm and 40 cm long, in particular between 20 cm and 30 cm, preferably between 20 cm and 25 cm.

The insertion sleeve can be made of stainless steel, medical-grade plastic material, titanium, polyether ether ketone, and/or the like. In some embodiments, the insertion sleeve is intended for single use, or is a single-use product. The insertion sleeve may have a friction-reducing coating on its inner surface-for example, Teflon, silicone, and/or the like.

A wall thickness of the insertion sleeve can, for example, be between 0.5 mm to 1.5 mm.

The insertion sleeve can be substantially homogeneous along its longitudinal extension. This may mean that the cross-section of the insertion sleeve substantially does not vary along its longitudinal extension, particularly along a region intended for arrangement within the ureter. A proximal portion of the insertion sleeve may have a different cross-section. In particular, however, the inner contour of the insertion sleeve, which defines, for example, the guide channel, is homogeneous along the longitudinal extension of the insertion sleeve. As already described, the outer contour can differ, in particular at the proximal end.

The oval shape of the cross-section can refer to the outer contour of the insertion sleeve and/or the inner contour of the insertion sleeve. In particular, the cross-section can be oval-ring shaped, or the insertion sleeve can have an oval cross-section in the form of an oval ring and/or an oval, ring-shaped cross-section. An oval ring or “oval ring-shaped” can mean to a shape that, analogously to a circular ring, has an oval outer contour and an oval hole. Accordingly, the cross-section of the insertion sleeve can have an opening, in particular an oval opening. The cross-section can have an elongated shape and in particular define two axes of different lengths, which lie perpendicularly to one another and perpendicularly to the longitudinal axis of the insertion sleeve. In this respect, the cross-section can be asymmetrical. In some embodiments, the cross-section has an elliptical shape, or the insertion sleeve has an elliptical cross-section. It goes without saying that, in the aforementioned embodiments, the wall thickness can vary in the circumferential direction and/or in the longitudinal direction of the insertion sleeve.

The cross-section can be defined by a first axis and a second axis that are perpendicular to one another and perpendicular to the longitudinal axis of the insertion sleeve, wherein the second axis is longer than the first axis. The second axis can, for example, be longer by a factor of up to 1.2, in particular up to 1.5, preferably up to 2. The inner contour and the outer contour of the insertion sleeve can have a different or the same shape, wherein the inner contour and outer contour each have an oval, in particular elliptical, shape. In some embodiments, the wall thickness of the insertion sleeve in the circumferential direction is substantially homogeneous.

The second axis can have a length of, for example, up to 12 mm, in particular up to 10 mm, preferably up to 8 mm. The first axis can have a length of, for example, up to 10 mm, in particular up to 8 mm, preferably up to 6 mm. In principle, a shorter length is desirable in order to minimize the strain on the patient.

A cavity can refer to both the bladder and the urethra. In some embodiments, the insertion sleeve is configured to be positioned in the urethra in such a way that a distal portion of the insertion sleeve protrudes into the bladder. In some embodiments, however, the insertion sleeve may also be, in particular, slightly shorter than the urethra of the treated patient, or may not extend completely to the bladder neck when positioned in the urethra.

“Distal” should be understood to mean, in particular, facing a patient during operation and/or facing away from an operator and/or user during operation. In particular, proximal is the opposite of distal. “Proximal” should be understood to mean, in particular, facing away from a patient and/or facing an operator and/or user during operation.

An “elongated part” is to be understood in particular as a component whose main extension is at least a factor of five, preferably at least a factor of ten, and particularly preferably at least a factor of twenty larger than a largest extension of the component perpendicular to its main extension, i.e., in particular a diameter of the component. A “main extension” of a component should be understood in particular as its longest extension along its main extension direction. A “main extension direction” of a component is to be understood to mean, in particular, a direction extending parallel to a longest edge of a smallest imaginary cuboid which only just fully encloses the component and which preferably passes through a geometric center and/or a center of mass of the component.

The shaft can, for example, comprise an elongated, cylindrical, in particular hollow-cylindrical, element that carries instruments, apparatuses, and/or the like, by means of which a resection procedure can be performed. Said shaft can facilitate the insertion of the instruments, apparatuses, and/or the like into the cavity, in particular the bladder. In general, access to the cavity can thus be achieved via the shaft. The shaft can be held and/or manipulated by a user at its proximal portion to align the instruments, apparatuses, and/or the like within the cavity. The manipulation in particular also allows the user to perform the resection procedure. The shaft can accordingly be rigid and/or bend-proof. It can have a length of, for example, 10 cm to 50 cm, in particular 15 cm to 40 cm, preferably 20 cm to 30 cm.

The shaft can define different shaft portions along its main extension direction or along its longitudinal extension along the longitudinal axis, in particular and/or at least the first shaft portion and the second shaft portion. The first shaft portion may have a different outer contour than the second shaft portion. Furthermore, a third shaft portion may be provided, which in particular also has a different outer contour. The first shaft portion and the second shaft portion can carry different functional units. A functional unit may include, for example, an imaging device, an actuating device, a supply device, and/or the like. A supply device may include, for example, supply lines, electrical supply lines, a power transmission element, and/or the like. The supply device can accordingly be provided to connect a distal functional unit to a proximal shaft portion and supply power thereto. The shaft can occupy a small amount of installation space, in particular in a portion that carries the supply device. In some embodiments, the supply device is arranged in the first shaft portion.

In general, the cross-section of the first shaft portion can have a different, in particular smaller, circumference than the second shaft portion. The outer contour of the first shaft portion, in particular the cross-section of the first shaft portion, can differ from the outer contour of the second shaft portion, in particular the cross-section of the second shaft portion.

The shaft may have a distal end piece that is arranged on a distal shaft portion. In particular, the shaft can be fed into the cavity of the patient in such a way that the distal end piece is arranged within the cavity. The distal end piece can carry functional units used to perform the resection procedure. The distal end piece can define a distal end portion of the shaft and extend over up to 5 cm, in particular up to 4 cm, preferably up to 3 cm, of the shaft, for example.

The shaft can be movable, in particular by a user, independently of and relative to the insertion sleeve. For a resection, the user can insert the insertion sleeve together with the shaft and the resection tool into the urethra. This can also happen sequentially, for example, if the different modules are of different lengths. The term “together” can be understood to mean that the insertion can be a single process and the user does not have to grasp the individual modules one after the other and advance them individually through the urethra. When the insertion sleeve is partially fed into the urethra or cavity, the user can continue advancing the shaft, in particular to explore the bladder and/or to perform a resection procedure.

In other words, the shaft can be configured to be moved within the insertion sleeve after being fed into the cavity, in particular when removing tissue. In contrast to a conventional resection procedure, the removal of tissue thus does not involve moving an assembly that is in direct contact with the urethra. Instead, the shaft is moved relative to the insertion sleeve, while the insertion sleeve remains stationary relative to the urethra.

According to the above explanations, the shaft can have different positions, which can be defined by a different longitudinal position of the shaft relative to the insertion sleeve. Different shaft portions can be arranged within the insertion sleeve in different longitudinal positions. For example, in a first longitudinal position, the first shaft portion can be at least partially arranged inside the insertion sleeve. In a second longitudinal position, the second shaft portion can be at least partially arranged inside the insertion sleeve. In the second longitudinal position, the application configuration can be defined. Furthermore, there may be other longitudinal positions in which an application configuration is defined.

An application configuration can mean a relative arrangement of components and/or assemblies of the resectoscope device in which a resection procedure can be performed. For example, functional units necessary for the resection procedure, such as a tool tip, can be arranged inside the bladder and be movable within it, in particular in three spatial directions and/or rotatable about the longitudinal axis of the shaft. In particular, in the application configuration, a tool tip of the resectoscope device is rotatable about the longitudinal axis of the shaft. The tool tip can be rotatable by rotating the first shaft portion in the insertion sleeve.

Furthermore, in the application configuration, the first shaft portion can be linearly movable within the insertion sleeve. This can mean that the shaft can be advanced independently of the insertion sleeve. Accordingly, the shaft can be positioned inside the bladder.

The phrase “to be rotatable within the insertion sleeve” can be understood to mean that the first shaft portion is rotatable about a longitudinal axis of the insertion sleeve, the first shaft portion, and/or the shaft. It can therefore be rotated in the insertion sleeve in a state where the insertion sleeve is arranged and/or fixed in the urethra. The first shaft portion can accordingly be rotatable independently of the insertion sleeve. This can mean that the insertion sleeve remains substantially stationary during rotation of the first shaft portion.

The second shaft portion can be rotatable by rotating the first shaft portion about the longitudinal axis of the shaft. The first shaft portion and the second shaft portion can therefore be connected to one another for conjoint rotation.

The phrase “to be arranged outside the insertion sleeve” can be understood to mean that a longitudinal axis of the second shaft portion and a longitudinal axis of the insertion sleeve do not intersect. The second shaft portion can accordingly be arranged distally or proximally to the insertion sleeve.

According to some embodiments, the second shaft portion in at least one direction that is transverse to the longitudinal axis of the shaft has an extension which is greater than the diameter of the largest circle inscribable in the oval cross-section of the insertion sleeve. Accordingly, the second shaft portion, when it is arranged inside the insertion sleeve, cannot be rotated by at least 180°. The insertion sleeve and the second shaft portion can accordingly collectively form a rotational locking unit. Furthermore, it follows that the second shaft portion can only be inserted into the insertion sleeve in certain rotational positions relative to said sleeve. These specific rotational positions can be referred to as “rotational insertion positions.” In the aforementioned direction, the shaft can have an outer diameter of, for example, up to 11 mm, in particular up to 9 mm, preferably up to 7 mm. The “largest circle inscribable in the oval cross-section of the insertion sleeve” can mean a circle that can be inscribed in the, in particular oval, opening of the cross-section of the insertion sleeve. Accordingly, the smallest distance from the center of the cross-section to the inner contour of the cross-section can correspond to the radius of the largest circle inscribable in the oval cross-section of the insertion sleeve.

In some embodiments, the second shaft portion has an extension transverse to the longitudinal axis of the shaft that is larger than the diameter of the largest circle inscribable in the oval cross-section of the insertion sleeve such that the second shaft portion cannot be inserted into the insertion sleeve in any rotational position relative to the insertion sleeve.

Available installation space can be utilized efficiently if, at least in a feed configuration, the second shaft portion is arranged in the insertion sleeve and mounted such that it cannot rotate. The second shaft portion can therefore be designed in such a way that it cannot rotate when arranged in the insertion sleeve. Accordingly, the insertion sleeve and the second shaft portion can collectively form a locking device. The insertion sleeve can accordingly serve not only to reduce friction, but also to simply mount the shaft such that it cannot rotate. In the feed configuration, the insertion sleeve can be introduced into the cavity, in particular the ureter, together with the shaft. Mounting the shaft such that it cannot rotate can be advantageous, since it allows for good control over the shaft and functional units located thereon.

In some embodiments, the shaft can be immovably connected to the insertion sleeve in at least one feed configuration. Linear displacement may be prevented in the feed configuration. The shaft may comprise, for example, a releasable fixing mechanism designed to optionally fix the insertion sleeve.

The feed configuration and the application configuration can optionally be set by a user by displacing the shaft relative to the insertion sleeve along the longitudinal axis of the shaft. The user can therefore set either the feed configuration or the application configuration by manipulating the shaft. These two configurations can therefore be set relatively easily. No complex mechanisms are needed to achieve the corresponding configurations. The resectoscope device can therefore be designed to be simple and to save installation space. The user can therefore selectively set the shaft to be mounted such that it can or cannot rotate by means of the relative, in particular linear, displacement, or the shaft can be set to be mounted such that it can or cannot rotate by means of the relative, in particular linear, displacement.

The functionality of the insertion sleeve can be extended if a return for a rinsing fluid is formed between the insertion sleeve and the first shaft portion. The return can be open in the application configuration and closed in the feed configuration. Accordingly, the installation space can be used efficiently, and the overall size of the insertion sleeve can be kept compact. Fluid can be drawn from the bladder via the return. For example, rinsing fluid could be introduced into the bladder using a rinsing device of the resectoscope device. This is necessary, for example, to fill and stretch the bladder. In this state, tissue can be processed and removed more easily. Furthermore, tissue remnants produced during the resection procedure can be drawn out. Conventional resectoscopes offer at best a return with a small diameter, which is insufficient for efficient bladder rinsing. If a circular insertion sleeve is used, the return could be arranged, for example, between the shaft, which is also circular, and the insertion sleeve. However, this return also offers a small overall volume flow. The inventors recognized that, by using the insertion sleeve with an oval cross-section and the shaft with the different shaft portions, there is available installation space within the insertion sleeve in the application configuration. This can accordingly be used efficiently for a high-performance return that offers a large volume flow. This can be opened in the manner described above by the relative, in particular linear, displacement of the shaft. This is achieved specifically by the fact that the second shaft portion can have an outer contour with a larger circumference than the first shaft portion. A closed return can be understood as one whose inlet is largely covered. The inlet thus does not necessarily have to be completely covered.

As already described, the shaft and/or the resectoscope device may comprise a rinsing device used to rinse a region that can be worked on by the resectoscope device, in particular by a resection tool of the resectoscope device. During tissue ablation, the ablated tissue can be washed away, ensuring a good visibility of the tissue being treated. The rinsing device may comprise a channel that is incorporated into the shaft and/or extends along the longitudinal axis of the shaft. Other types of rinsing devices are also conceivable-for example, a rinsing device that is integrated into a resection tool. This allows rinsing fluid to be introduced proximally into the channel and guided along the shaft into the cavity. The rinsing device may comprise an outlet on a distal shaft portion through which rinsing fluid can leave the rinsing device. The distal

The shaft or the first shaft portion can be rotatable by 180°in the application configuration if the first shaft portion in no direction that is transverse to the longitudinal axis of the shaft has an extension which is greater than the diameter of the largest circle inscribable in the oval cross-section of the insertion sleeve. Accordingly, a shaft portion arranged in the cavity can be flexibly positioned without restricting a rotary angle around the longitudinal axis.

According to some embodiments, in particular in the application configuration, the first shaft portion in the insertion sleeve is guided linearly along the longitudinal axis of the shaft. This gives the user great control over the shaft and allows them to position it precisely within the bladder. When the shaft is moved linearly for tissue removal, the linear guidance allows the user to perform the tissue removal movement precisely. For linear guidance, the first shaft portion can at least partially contact the inner contour of the insertion sleeve. Accordingly, the radial mobility of the first shaft portion in the insertion sleeve is restricted.

In some embodiments, the first shaft portion is radially immovably accommodated within the insertion sleeve. This can be achieved, for example, if the first shaft portion and the insertion sleeve collectively form a fit. In particular, the first shaft portion has an outer contour which at least partially coincides with a circular outer cross-section having a first diameter that is of such a size that the first shaft portion is fitted into the largest circle inscribable in the oval cross-section of the insertion sleeve. The circular outer cross-section having the first diameter can be an imaginary outer cross-section. The imaginary outer cross-section can accordingly describe and/or define portions of the outer contour of the first shaft portion. The first shaft portion can, for example, define at least one circular arc along portions of the circumference, which is designed to contact the inner contour of the insertion sleeve.

In some embodiments, the insertion sleeve has an elliptical cross-section. The major axis of the ellipse can substantially correspond to the diameter of the shaft. The shaft has in particular a substantially circular cross-section.

Together with the first shaft portion, the insertion sleeve can therefore simultaneously define a linear guide device, in particular the fit, and the return. The installation space is used efficiently, and an insertion sleeve with a small circumference can be provided. This is made possible by the oval cross-section of the insertion sleeve.

Furthermore, the first shaft portion can be arranged proximally to the second shaft portion. Accordingly, the aforementioned features may refer to the fact that the second shaft portion is arranged within the bladder in the application configuration. In the application configuration, a portion of the shaft within the bladder can therefore be rotatable by rotating the first shaft portion in the insertion sleeve about the longitudinal axis of the shaft.

According to some embodiments, the second shaft portion at least partially defines a distal end piece of the shaft. Alternatively or additionally, the distal end piece has the second shaft portion. In other words, the shaft can have a distal end piece that partially forms the second shaft portion. The second shaft portion can, for example, define a distal end portion of the distal end piece. The distal end piece may accordingly have the aforementioned features of the second shaft portion. Consequently, in the feed configuration, the distal end piece can be mounted in the insertion sleeve such that it cannot rotate. In this case, the shaft thus does not extend substantially distally beyond a distal end of the insertion sleeve. Only part of the distal end piece might protrude distally. This can prevent the shaft from being accidentally pushed too far into the bladder during insertion and injuring the bladder.

The distal end piece may have a larger outer contour than the first shaft portion. Accordingly, the distal end piece can provide a greater amount of installation space for arranging functional units. The inventors recognized that essential functional units of the resection device must be arranged in particular on the distal end piece, since this is inserted into the bladder. Accordingly, a lot of installation space is required at the distal end piece. However, less installation space is required proximal to the distal end piece, especially along the first shaft portion, since only electrical lines, supply lines, etc., for the functional units of the distal end piece run here, for example. Using the oval cross-section makes use thereof, and the overall circumference of the insertion sleeve can be reduced. The distal end piece can therefore substantially fill an opening in the cross-section of the insertion sleeve, thus efficiently utilizing the installation space. The inventors recognized that a substantially circular cross-section does not necessarily have to be used for the distal end piece. In some embodiments, as will be described below, an elongated cross-section can even be advantageously used for the functional units.

The installation space can be utilized efficiently if the second shaft portion comprises an image acquisition apparatus that defines an observation region. The image acquisition apparatus regularly requires the largest proportion of installation space in the shaft. This can therefore be provided in the distal end piece or on a shaft portion with a relatively large amount of available installation space. As already described, together with the insertion sleeve this portion can additionally provide rotationally fixed support, in particular in the feed configuration. Therefore, the installation space required for the image acquisition apparatus can be used to achieve another function. Overall, this allows the resectoscope device to be compact.

Endoscopic imaging can be performed using the image acquisition apparatus when it is located on the distal end piece of the shaft. The image acquisition device can be configured to image objects within the observation region, in particular the interior of the cavity, and/or to generate image data. An object can, for example, comprise tissue, in particular a portion of the bladder wall. The image data can be used to generate a representation of the interior of the cavity on a display device. The display device allows the user to observe the inside of the bladder and the progress of the resection or ablation of tissue. The image acquisition device can be positioned within the cavity independently of the resection tool, in particular by the user.

According to some embodiments, the image acquisition apparatus may comprise at least one image acquisition unit which is formed by at least one input optics and/or at least one image sensor for generating images. The at least one input optics can be arranged in particular on a distal end face of the shaft, in particular the second shaft portion and/or the distal end piece. Accordingly, the resectoscope device can be compact and enable endoscopic imaging. The inlet optics can be precisely positioned within the bladder by means of the linear guidance and the rotatable mounting of the first shaft portion in the insertion sleeve. In particular, these features allow the inlet optics to be kept stable during the resection procedure and enable high-quality imaging to be performed. The image sensor can be configured as a CCD chip and/or a CMOS chip, for example. The image acquisition unit can define a modular assembly of the image acquisition unit.

The distance between the tool tip and the tissue can be effectively estimated and the user can effectively assess the anatomy if the at least one image acquisition unit is configured for stereo imaging. The resection procedure can be performed particularly precisely and gently. In addition, the user can better orient themselves within the space or inside the bladder and position various functional units more precisely relative to the tissue. Stereo imaging can be achieved, for example, by capturing two spatially offset images of an object, thus allowing for a three-dimensional representation of the object. This involves taking pictures from different viewpoints, for example, which are then combined to create a spatial overall image. This allows, for example, for the generation of depth information, which can be used for the precise measurement of structures and/or distances within the image field recorded.

The inventors have recognized that, especially with stereo imaging, the use of an asymmetric distal end piece is advantageous for the efficient use of the installation space. Since two lenses are usually arranged side by side, a greater extension may be necessary in a direction that is transverse to the longitudinal axis of the shaft than in a direction perpendicular thereto that is perpendicular to the longitudinal axis. Therefore, using a circular cross-section for the distal end piece would result in it having an unnecessarily large circumference. Accordingly, the oval shape of the cross-section of the insertion sleeve can take this circumstance into account. By using the two different-shaped shaft portions, effective positioning can still be provided and/or the return can be formed.

The image acquisition unit can accordingly have a stereo input optics with two objectives arranged side by side, wherein the sum of the diameters of the objectives is greater than the diameter of the largest circle inscribable in the oval cross-section of the insertion sleeve. The objectives can comprise, for example, a lens, in particular a converging lens, and/or a lens arrangement. They can be arranged side by side in the direction in which the cross-section of the distal end piece and/or the second shaft portion has the greatest extension. They do not necessarily have to be arranged precisely on the correspondingly largest axis of the cross-section. Since the sum of the diameters of the objectives is larger than the diameter of the largest circle inscribable in the oval cross-section of the insertion sleeve, the distal end piece is only mounted within the insertion sleeve to a limited extent and/or such that it cannot rotate. Furthermore, as already described, the installation space can be utilized efficiently.

A large observation region can be provided if the image acquisition apparatus comprises at least two image acquisition units directed in different viewing directions. Each image acquisition unit can be formed by an input optics and/or an image sensor for generating images, in particular a CCD chip or CMOS chip.

The at least two image acquisition units can in particular comprise a first image acquisition unit and a second image acquisition unit, wherein the first image acquisition unit is directed in a distal viewing direction, and wherein the second image acquisition unit is directed in a proximal viewing direction. The viewing direction from two different sides further improves the image available to the user through the images taken from different perspectives, and in particular improves visibility when setting a starting point for tissue removal.

In some embodiments, the image acquisition device comprises exactly two, exactly three, and/or more or at least two image acquisition units directed in different viewing directions, the image acquisition units together defining a continuous observation region. The observation sub-regions collectively define the observation region and can in particular overlap.

The viewing direction can be directed in a proximal direction with respect to the shaft. The viewing direction can be directed distally with respect to the shaft. The viewing direction can be defined by a viewing angle relative to the longitudinal axis of the shaft. A viewing angle of 0° to less than 90° can be interpreted as directed in a distal viewing direction, and a viewing angle of more than 90° to 180° can be interpreted as directed in a proximal direction.

The image acquisition device can comprise at least three image acquisition units directed in three different viewing directions. The arrangement of multiple image acquisition units with different viewing directions improves the imaging of tissue to be removed that is available to a user. The at least three image acquisition units can define three different observation sub-regions, in particular one observation sub-region each, wherein the different observation sub-regions can at least partially overlap. The observation sub-regions can therefore each have a portion that lies within an adjacent observation sub-region. In the case of overlapping observation sub-regions viewed from different viewing directions, the image of tissue to be removed that is available to the user is further improved.

The overlapping observation sub-regions can collectively define the observation region. The multiple image acquisition units can be arranged offset in a longitudinal direction and/or with respect to a longitudinal axis of the shaft, in particular the distal end piece of the shaft, and in particular arranged one behind the other.

The one or more image acquisition unit(s) can each be formed by camera modules, in particular camera modules having short-profile objectives or so-called camera cubes with wafer-level optics. The one or more image acquisition units each have, in particular, an individual viewing angle. The individual viewing angle can, for example, be 60°, 65°, 70°, 75°, 80°, 85°, 90°, 95°, 100°, 105°, or 110°.

The viewing direction of an image acquisition unit can be a primary viewing direction of the relevant image acquisition unit. For example, the viewing direction can be a central axis of an input optics of the relevant image acquisition unit. The individual viewing angle can be an angle that is spanned relative to the viewing direction. In particular, the central axis can be an angle bisector of the individual viewing angle. Depending upon the design of the image acquisition units, this can be defined by two individual viewing angles that are perpendicular to each other. These can, for example, be defined as viewing angles relative to a transverse axis or viewing angles relative to a vertical axis, each perpendicular to the central axis and/or the viewing direction. In particular in cases where an image acquisition unit comprises a rectangular image sensor, both viewing angles belonging to the image acquisition unit can be assigned to a short and a long side of the rectangular image sensor.

The specification of an individual viewing angle can also be understood as the largest viewing angle of the relevant image acquisition unit and/or as a viewing angle that corresponds to a diagonal of the relevant image sensor. An image acquisition unit can also be described by a horizontal viewing angle, a vertical viewing angle, and a diagonal viewing angle. Specifications relating to a viewing angle can refer to any of these three variables and preferably refer to a diagonal viewing angle.

To illuminate the interior of the cavity, the imaging regions and/or the observation region, an illumination device may be arranged at a distal end piece, configured to provide illumination light, of the shaft. Illuminating the imaging region and/or the observation region supports the capture of an image of it with the image acquisition device and thus in particular increases the image quality of an image provided to the user. The illumination device may comprise a plurality of illumination units which are directed in particular in different illumination directions. The individual illumination units can be formed by light-emitting diodes and/or laser diodes and/or light guides.

A compact resectoscope device can be provided if the image acquisition device is integrally designed with the illumination device. In particular, the distal end piece can be designed not to occupy much space and to utilize installation space efficiently. In some embodiments, the image acquisition device and the illumination device can be designed in pairs. This can mean that each illumination unit is integrally designed with an image acquisition unit.

High patient comfort can be achieved and postoperative complications can be reduced; the resectoscope device comprises a resection tool, wherein the shaft and the resection tool are independently movable relative to the insertion sleeve along the longitudinal axis of the shaft. The shaft and the resection tool can be configured to be moved within the insertion sleeve after being fed into the cavity during a removal of tissue. The insertion sleeve can in particular be designed to rest atraumatically during tissue removal. It is understood that the insertion sleeve could move slightly relative to the ureter. However, this movement may be negligibly small, or significantly smaller than the relative movement between a shaft and the urethra during a conventional resection. In other words, the insertion sleeve can be configured to remain at least substantially stationary when the shaft is positioned within the cavity in relation to surrounding and, in particular, contacting tissue.

The resection tool can have a tool tip. This tool tip and/or the resection tool can be configured to remove tissue by cutting and/or vaporization. The resection tool, in particular the tool tip, may have an electrically conductive material, in particular a wire, through which an electric current can flow. The resection tool, in particular the tool tip, can be designed as an RF tool (radio-frequency tool). Furthermore, it may have a resection loop and/or be loop-shaped, hook-shaped, or spherical.

The resection tool, in particular the tool tip, can be moved independently of and/or together with the shaft for the tissue removal movement, both times independently of the insertion sleeve. The tool tip is moved back and forth, for example, while it contacts tissue to be removed. This tissue is accordingly removed layer by layer.

The resection tool, in particular the tool tip, may in particular have an RF resection loop (radio-frequency resection loop). Alternatively or additionally, the resection tool may comprise a laser for cutting and/or vaporization. The laser can be designed in particular for holmium laser enucleation or KTP laser vaporization, or can be configured as a continuous-wave laser. These laser methods differ in the wavelength and energy of the laser light used, and in particular in their energy effect in tissue and their penetration depth. Alternatively or additionally, the resection tool, in particular the tool tip, can have a monopolar or a bipolar RF tool (radio-frequency tool). The monopolar HF tool can be designed as a hook electrode, ball electrode, or spatula electrode for cutting and/or vaporization. The bipolar HF tool can be designed in particular as bipolar tweezers or forceps for cutting and/or vaporization. The resection tool can also be configured to perform contact coagulation on tissue. Depending upon the application, one of the described designs of the resection tool may be particularly suitable.

A safe and precise ablation movement can be achieved if the shaft comprises a guide device for longitudinal guidance of the resection tool. The guide device may, for example, comprise a guide groove that extends along the longitudinal axis. It may be embedded in the shaft, for example, on one upper side. Furthermore, the resection tool can comprise at least one carrier arm that is positioned proximally to the tool tip. The carrier arm can be guided and/or accommodated in the guide apparatus.

A space-saving, compact resectoscope device can be provided if the resection tool comprises two carrier arms and a tool tip arranged between the carrier arms, in particular an HF resection loop. The shaft can be positioned between the carrier arms. A carrier arm can be configured to connect the tool tip to a proximal module, in particular the operating module. If the tool tip is electrically operated, an electrical supply line, e.g., a cable, may be guided within the carrier arm, and/or the carrier arm may form such a supply line. In some embodiments, the carrier arm can be shaped approximately like a tube and accommodate at least one further functional unit and/or further supply lines. Furthermore, a carrier arm can be designed to be rigid and/or mechanically stable. For example, the tool tip can be moved distally relative to the shaft. In a distally advanced state, the resection tool can, for example, extend up to 10 cm beyond a distal end of the shaft. In this case, the carrier arm is designed to be rigid and/or stable and/or stiff enough that the tool tip does not bend or flex significantly.

The resectoscope device may further comprise a proximal operating assembly which, as a whole, is movable relative to the insertion sleeve. The insertion sleeve can therefore remain stationary and/or move only minimally when the proximal operating module is moved. The shaft and/or the resection tool can, for example, be coupled proximally to the operating module. By moving the operating module back and forth, the user can move the shaft and the resection tool, in particular jointly, relative to the insertion sleeve and, in particular, perform an ablation movement. The proximal operating assembly can therefore be gripped and/or manipulated by a user, wherein the user can position the shaft portion arranged within the bladder, in particular the distal end piece, by manipulating the proximal operating assembly.

The shaft and the resection tool can therefore be movable independently of one another and/or can be moved independently of one another, in particular by a user, wherein the movement is carried out relative to the insertion sleeve.

In some embodiments, the proximal operating module further comprises an actuating mechanism by means of which the resection tool can be moved along the longitudinal axis independently of the shaft. By actuating the actuating mechanism, the user can move the resection tool back and forth, in particular independently of the shaft and/or the insertion sleeve, in particular to perform an ablation movement.

In other words, the shaft and the resection tool can be configured to be moved within the insertion sleeve after being inserted into the cavity during the ablation of tissue. In contrast to a conventional resection, the ablation of tissue does not involve moving a module that is in contact with the urethra.

The insertion sleeve can be detachably coupled to the operating module.

For example, the operating module together with the insertion sleeve can form a coupling mechanism configured to detachably connect the insertion sleeve to the operating module. If necessary, the insertion sleeve can be released-for example, after being inserted into the cavity together with the shaft and the resection tool. The insertion sleeve can also be mechanically recoupled to the operating module if required in order to remove the insertion sleeve together with the shaft and the resection tool from the cavity.

### BRIEF DESCRIPTION OF THE DRAWINGS

The present disclosure will be described by way of example below with reference to the accompanying figures. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example can be transferred accordingly to the other examples of the object. If objects are named using number words, such as first, second, third object, etc., these are used to name and/or assign objects. Accordingly, for example, a first object and a third object may be included, but not a second object. However, a number and/or sequence of objects could also be derived using numerical words.

FIG. 1 is a schematic representation of a resectoscope device in a side view;

FIG. 2 is a schematic representation of a resectoscope, comprising the resectoscope device, a control unit, and a display apparatus.

FIG. 3 is a schematic plan view of the resectoscope device in an application configuration;

FIG. 4 shows a cross-section of the resectoscope device in an application configuration in the region of the insertion sleeve;

FIG. 5 shows a schematic representation of an insertion sleeve and the largest circle inscribable in an oval cross-section of the insertion sleeve;

FIG. 6 is a schematic side view of the resectoscope device in a feed configuration;

FIG. 7 is a schematic plan view of the resectoscope device in the feed configuration; and

FIG. 8 is a schematic distal view of the resectoscope device in the feed configuration.

### DETAILED DESCRIPTION OF PREFERRED EMBODIMENTS

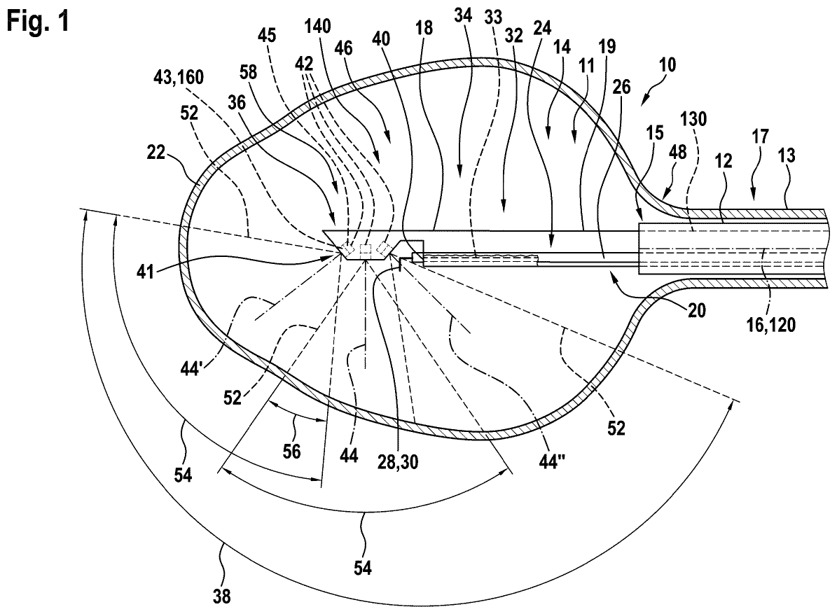

FIG. 1 is a schematic representation of a resectoscope device 10 in a side view. The resectoscope device 10 is configured to perform a resection and comprises an insertion sleeve 12 having a longitudinal axis 16, a shaft 18, and a resection tool 20. The resectoscope device 10 is shown in one of many possible positions, specifically in a position that defines an application configuration. In this position, the shaft 18 is mounted in the insertion sleeve 12 so as to be rotatable and linearly guided. The positions are defined by a relative position of the insertion sleeve 12, the shaft 18, and the resection tool 20 to each other. These modules 12, 18, 20 can be moved independently of each other relative to each other.

The insertion sleeve 12 is shown in a state inserted into a urethra 13. More precisely, a first shaft portion 130 is shown in a state in which it is accommodated in the insertion sleeve 12. A distal end piece 15 of the insertion sleeve 12 protrudes into a bladder 11. It is also conceivable that in other situations, e.g., depending upon an anatomy of a patient, the insertion sleeve 12 does not extend into the bladder 11. The insertion sleeve 12 has an oval cross-section, as will be described in more detail below. Furthermore, it is tubular and defines a guide channel 17. It thus provides trocar-like access to the bladder 11 for the shaft 18 and the resection tool 20.

The shaft 18 and the resection tool 20 extend along the longitudinal axis 16 of the insertion sleeve 12 through the guide channel 17 into the cavity 14, in this case the bladder 11. These modules 18, 20 can be inserted into the urethra 13 together with the insertion sleeve 12 by being mechanically coupled to each other. As will be explained below, the shaft 18 and the insertion sleeve are preferably collectively inserted into the urethra in a feed configuration. In this feed configuration, the second shaft portion 140 is arranged inside the insertion sleeve 12 and is mounted therein such that it cannot rotate. Once the insertion sleeve 12 is positioned in a desired position, the shaft 18 and the resection tool 20 can be advanced further distally. This allows the resectoscope device 10 to be transferred from the feed configuration to an application configuration. If the shaft 18 is moved distally in such a way that the first shaft portion 140 is no longer arranged within the insertion sleeve 12, the shaft 18 can be rotated about its longitudinal axis 120.

The shaft 18 has a distal end piece 34 which is immovably connected to a main body 19 of the shaft 18. The distal end piece 34 has the second shaft portion 140. The main body 19 has the first shaft portion 130. The shaft 18 has an image acquisition apparatus 36 on the distal end piece 34 for endoscopically imaging the interior of the bladder 11 and an illumination apparatus 46 for illuminating the interior of the bladder 11. Accordingly, the image acquisition apparatus 36 is arranged in the second shaft portion 140. The image acquisition device 36 and the illumination device 46 are designed integrally together. The image acquisition device 36 comprises three image acquisition units 42, which define different viewing directions 44, 44′, 44″. The image acquisition units 42 also each have a viewing angle 52. One image acquisition unit 42 has a viewing direction 44′ toward the distal and another image acquisition unit 42 has a viewing direction 44″ toward the proximal. The image acquisition units 42 are each formed by at least one input optics 43 and/or at least one image sensor 45 for generating images (shown only by way of example on one of the image acquisition units). One of the input optics 43 of the image acquisition units 42 is arranged on a distal end face 41 of the shaft 18, in particular the distal end piece 34 and/or the second shaft portion 140. The image acquisition units 42 together define a continuous observation region 38. Each of the image acquisition units 42 accordingly has its own observation sub-region 54 according to each viewing angle 52. Each of these has at least one overlapping region 56 that partially overlaps an observation sub-region 54 of an adjacent image acquisition unit. The image acquisition units 42 each have a stereo input optics 160 with two objectives arranged side by side (only one is provided with a reference sign by way of example). This will be discussed in more detail below. The image acquisition units 42 are integrally designed with illumination units (not shown in detail) of the illumination device 46, which are designed as LED's.

The resection tool 20 comprises at least one carrier arm 26 and a tool tip 28, which is designed as an HF resection loop. More precisely, the resection tool 20 comprises two carrier arms 26. A guide apparatus 24 is formed on a surface of the shaft 18, which device is designed as a guide groove for the carrier arm 26. The guide apparatus 24 extends along the majority of the shaft 18. The end of the guide apparatus 24 is provided as an open guide groove. The guide device 24, in particular the guide groove, partially incorporates the carrier arms 26 of the resection tool 20. This ensures that the carrier arms 26 are guided linearly in the guide apparatus 24 along a longitudinal axis 120 of the shaft 18. The longitudinal axis 120 is arranged coaxially with the longitudinal axis 16. The shaft 18 and the resection tool 20 are movable independently of one another along the longitudinal axis 120 of the shaft 18. The carrier arms 26 can therefore be moved linearly from proximal to distal and vice versa in the guide device 24. This movement allows a tool tip 28 to be moved linearly relative to the shaft 18 and independently thereof. In particular, the tool tip 28 can be moved beyond a distal end 58 of the shaft 18.

Furthermore, the shaft 18 comprises a rinsing device 32, which comprises a channel 33 and an outlet 40. The channel 33 extends along the longitudinal axis 120 of the shaft 18, from proximal to distal, up to the distal end piece 34 of the shaft 18. A rinsing fluid can be conveyed through channel 33, exiting at the outlet 40. The rinsing fluid can be used to fill the bladder 11 for resection and also to rinse away tissue residues that arise during the resection.

In addition, a return 48 is formed between the insertion sleeve 12 and the first shaft portion 130, through which rinsing fluid can be transported out of the bladder 11. For example, a suction device (not shown) can be proximally arranged on the insertion sleeve 12.

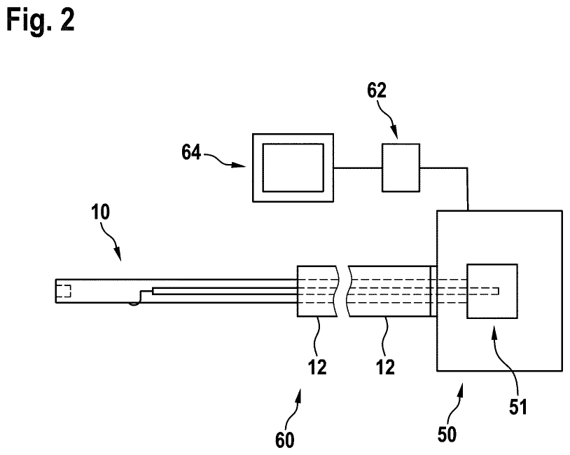

Furthermore, the resectoscope device 10 comprises a proximal operating assembly (see FIG. 2), which is movable as a whole relative to the insertion sleeve 12. A user can hold the resectoscope device 10 by the operating assembly and operate a resectoscope comprising the resectoscope device 10 (see FIG. 2).

Using the resectoscope device 10, a resection can be performed by the user as follows. First, he guides the shaft 18 and the resection tool 20 together with the insertion sleeve 12 into the cavity 14, in this case the urethra 13 and partly the bladder 11. He then brings the insertion sleeve 12 into a desired position within the urethra 13. Now, he fills the bladder 11 using the rinsing device 32 to open it up. The user then positions the distal end piece 34 or the second shaft portion 140 of the shaft 18 comprising the image acquisition apparatus 36 within the bladder 11. To do this, he pushes the shaft 18 distally relative to the insertion sleeve 12. The insertion sleeve 12 remains in its original position.

During the advancement movement, the resection tool 20 is coupled to the shaft 18. This means that both modules 18 and 20 are advanced together. By pivoting the shaft 18 and, if necessary, advancing it, the user can gain an overview of the interior of the bladder 11 and examine the tissue 22 on an inner wall of the bladder 11. This allows him to identify tissue that needs to be ablated, such as tissue that shows a pathological change. An example of such tissue is cancerous tissue.

This identified tissue can then be ablated using the tool tip 28. To do this, the user brings the distal end piece 34 of the shaft 18 into close proximity to the tissue by pivoting the shaft 18 and longitudinally positioning the shaft 18. During longitudinal positioning, the insertion sleeve 12 remains stationary relative to the urethra 13 or the cavity 14, as already described. In other words, the insertion sleeve 12 rests atraumatically.

After positioning the distal end piece 34, the user moves the resection tool 20 relative to the shaft 18, with the insertion sleeve 12 also remaining stationary and resting atraumatically, and brings the tool tip 28 into contact with the tissue. After activation of the tool tip 28, if it is designed as an HF resection loop, for example, the tissue can be removed layer by layer. For this purpose, the tool tip 28 is iteratively swept over the tissue, thereby superficially treating and ablating the tissue to be ablated. To sweep over the tissue, the resection tool 20 can be moved relative to the shaft 18. Furthermore, the shaft 18 can be moved together with the resection tool 20, while the insertion sleeve 12 remains stationary in the urethra 13.

During tissue ablation, the rinsing device 32 is used continuously, and rinsing fluid is transported out of the bladder 11 by means of the return 48 out of the bladder 11.

After resection is completed, the shaft 18 can be pulled out of the cavity 14 together with the resection tool 20 and the insertion sleeve 12.

The shaft 18 and the resection tool 20 are therefore configured to be moved within the insertion sleeve 12 after being inserted into the cavity 14 during the ablation of tissue 22. More precisely, the first shaft portion 130 is arranged in the insertion sleeve 12 during the resection procedure, is accommodated therein so as to be linearly guided, and is rotatably mounted therein. The insertion sleeve 12 is configured to rest atraumatically during the ablation of tissue 22.

FIG. 2 shows a schematic representation of the resectoscope 60, which comprises the resectoscope device 10. The resectoscope 60 is shown in a highly simplified form. Furthermore, the proximal operating module 50 can be seen and is movable as a whole relative to the insertion sleeve 12. The proximal operating module 50 also comprises an actuating device 51 by means of which the resection tool 20 can be operated, in particular moved forwards and backwards.

The resectoscope 60 is connected to a control unit 62, which can, for example, process image data and control a function of the resectoscope 60. In particular, the image acquisition device can be controlled by means of the control unit 62. The control unit 62 is connected to a display device 64, on which a representation of an endoscopic image can be generated. The representation allows the user to view the inside of the bladder 11 and thus monitor the resection procedure.

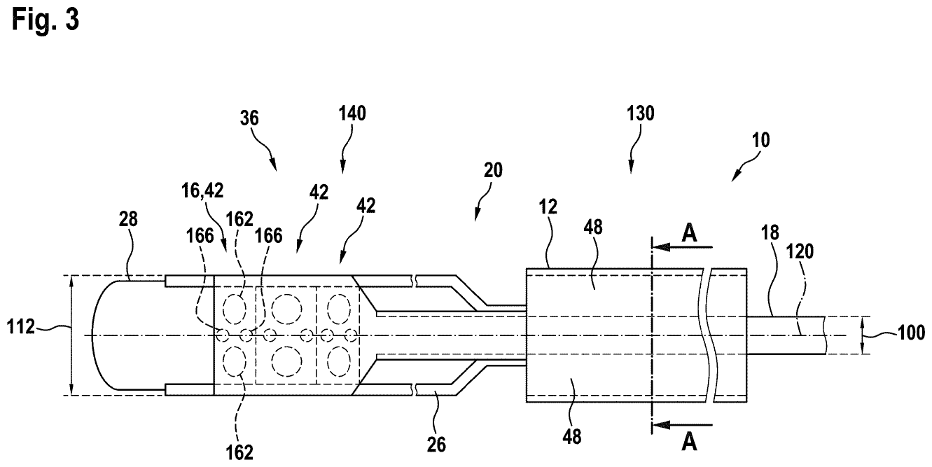

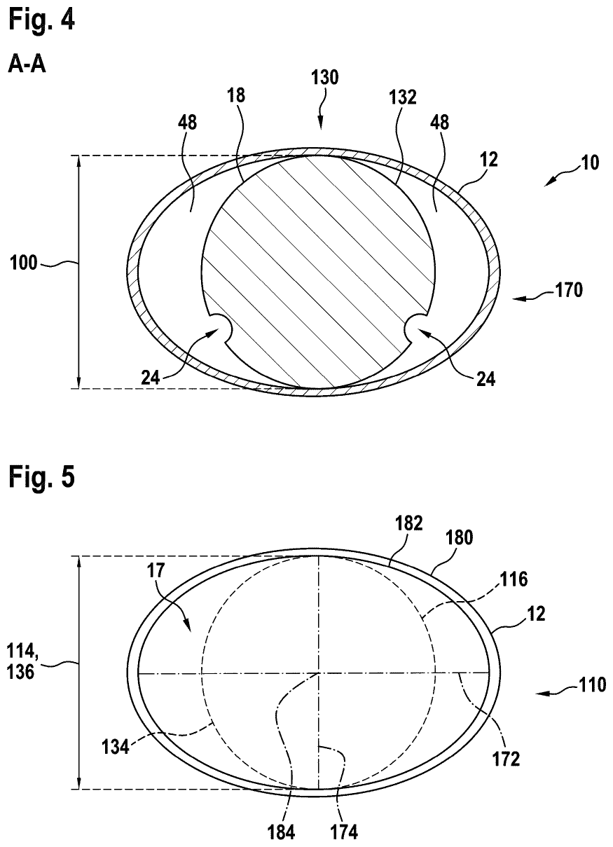

FIG. 3 and FIG. 4 show the resectoscope device 10 in an application configuration similar to the representation according to FIG. 1. The carrier arms are not shown. FIG. 3 shows a schematic plan view of the resectoscope device 10, and FIG. 4 shows a cross-section of the resectoscope device 10 in the region of the insertion sleeve 12. The second shaft portion 140 is arranged distally relative to the first shaft portion 130.

The second shaft portion 140 is arranged outside the insertion sleeve 12.

Said portion comprises the image acquisition apparatus 36, which has three image acquisition units 42. These are substantially identical and differ in terms of their viewing angle (see FIG. 1). Two objectives 162 are shown for each image acquisition unit 42, which are arranged side by side transversely to the longitudinal axis 120 of the shaft 180. These form the stereo input optics 160. Accordingly, the second shaft portion 140 comprises an image acquisition sensor (not shown) for each objective 162. By arranging the objectives 162 in pairs, stereo imaging can thus be performed in any viewing direction. Each image acquisition unit 42 also includes two LED's 166.

The second shaft portion 140 has in a direction that is transverse to the longitudinal axis 120 an extension 112 which is greater than the shaft diameter 100 of the shaft 18, in particular in the second shaft portion 130. The objectives 162 are arranged side by side in this direction of extension. Furthermore, it can be seen that a slot is formed between the insertion sleeve 12 and the first shaft portion 130 in the application configuration. This is used as the return 48. See also FIG. 4 for the return 48. This is formed on both sides, in particular in the direction of the major axis 172 of the insertion sleeve 12 (see FIG. 5) between the first shaft portion 130 and the insertion sleeve 12.

Furthermore, the carrier arms 26 can be seen, which widen laterally along the longitudinal axis 120. In a proximal portion, they are accordingly guided in the shaft (see FIG. 1). They are widened laterally at the distal end such that they can be guided laterally past the objectives 162. Accordingly, the tool tip 28 can be arranged distally relative to the shaft 18 or along the distal end piece 34, depending upon the position of the carrier arms 26 relative to the shaft 18. However, they cannot be guided along the first shaft portion 130, since the tool tip 28 is designed in such a way that it can be inserted into the insertion sleeve 12 together with the second shaft portion 140 in a state in which the tool tip 28 is arranged in a longitudinal position along the second shaft portion 140. The features relating to the resection tool 20 are to be understood as examples. Other configurations of a resection tool 20 are also conceivable.

As already described in connection with FIG. 1, the shaft 18, in particular the first shaft portion 130, in the application configuration is rotatably arranged in the insertion sleeve 12. Accordingly, the second shaft portion 140 can also be rotated by rotating the first shaft portion 130. The first shaft portion 130 is therefore arranged in the insertion sleeve 12 and is rotatable within the insertion sleeve 12. The second shaft portion 140 is arranged outside the insertion sleeve 12.

In FIG. 4, it can be seen that the shaft 18 has a circular cross-section 170 in the first shaft portion 130. In this figure, said shaft in no direction that is transverse to the longitudinal axis 120 of the shaft 18 has an extension which is greater than a diameter 114 of the largest circle 116 inscribable in an oval cross-section 110 of the insertion sleeve 12. The insertion sleeve 12 and the circle 116 are shown in more detail in a schematic representation in FIG. 5.

In this representation, the oval cross-section 110 of the insertion sleeve 12 can be seen. More precisely, the cross-section 110 has an elliptical ring shape. It therefore has an oval, in particular elliptical, opening that defines the guide channel 17. Furthermore, it has a homogeneous wall thickness that is substantially the same thickness along the circumference. The cross-section 110 is accordingly defined by an outer contour 180 and an inner contour 182, wherein both contours 180, 182 each have an oval, in particular elliptical, shape arranged concentrically. A major axis 172 and a transverse axis 174 coincide accordingly, wherein the major axis of the outer contour 182 accordingly is longer by twice the wall thickness. The major axis 172 corresponds to the longest connecting line between two points arranged on the inner contour 182, which passes through the center 184. The length of the transverse axis 174 of the elliptical inner contour 182 corresponds to the diameter 114 of the largest circle 116 inscribable in the oval cross-section 110 of the insertion sleeve 12. The diameter 114 therefore corresponds to the shortest connecting line between two points that lie on the inner contour 182 of the insertion sleeve 12, wherein the connecting line passes through the center 184. The cross-section 170 of the shaft 18 in the first shaft portion 130 (see FIG. 4) substantially corresponds to the circle 116.

As can be seen in FIG. 4, the first shaft portion 130 and the insertion sleeve 12 collectively form a fit 150, since the shaft diameter 100 substantially corresponds to the diameter 114 of the largest circle 116 inscribable in the oval cross-section 110 of the insertion sleeve 12. The shaft diameter 100 is slightly smaller to ensure linear mobility of the shaft 18 in the insertion sleeve 12. The first shaft portion 130 has an outer contour 132 which coincides with a circular outer cross-section 134 having a first diameter 136. The outer cross-section 134 corresponds to the circle 116 and the first diameter 136 corresponds to the diameter 114 of the circle 116. The first diameter 136 is of such a size that the first shaft portion 130 is fitted into the largest circle 116 that can be inscribed in the oval cross-section 110 of the insertion sleeve 12. This fit ensures that the first shaft portion 130 in the application configuration is guided linearly along the longitudinal axis 120 of the shaft 18 within the insertion sleeve 12. A radial position of the first shaft portion 130 is defined in the insertion sleeve 12, or the first shaft portion 130 is substantially immovable in the radial direction in the insertion sleeve 12. Rotation about the longitudinal axis 120 or about the center 184 is possible.

Furthermore, the guide apparatus 24 can be seen in FIG. 4, which is formed by two guide grooves in the first shaft portion 130.

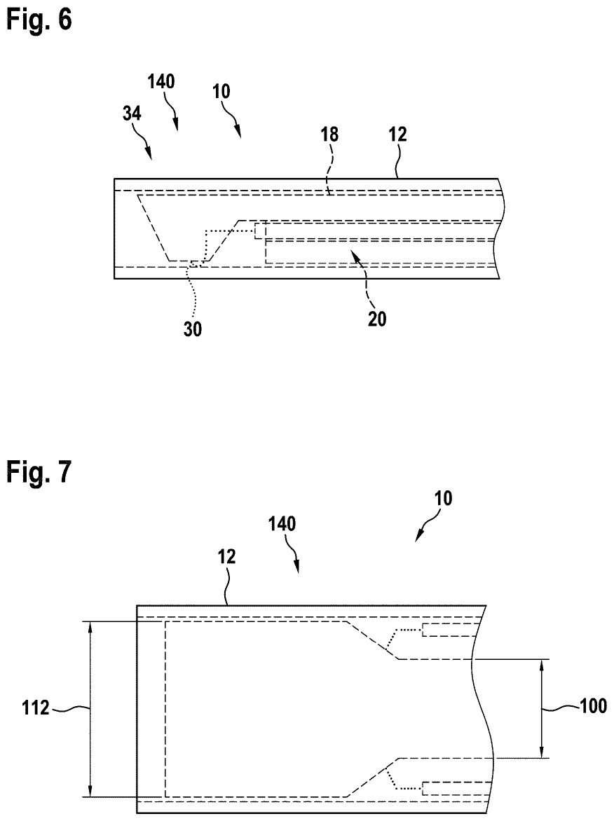

FIG. 6 shows a schematic side view of the resectoscope device 10 in another position. This position defines a feed configuration. In the feed configuration, the distal end piece 34 and the second shaft portion 140 of the shaft 18 are completely enclosed by the insertion sleeve 12. The resection tool 20 also does not extend distally beyond the insertion sleeve 12. In the position shown, the resectoscope device 10 could, for example, be inserted guided into the cavity 14. However, other positions are also conceivable in which at least one of the modules 18, 20 extends distally beyond the insertion sleeve 12. The HF resection loop 30 is placed around the underside of the second shaft portion 140.

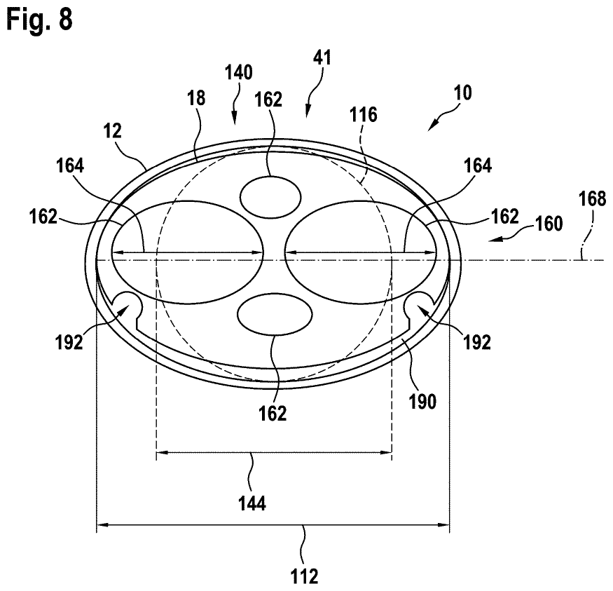

FIGS. 7 and 8 show the resectoscope device 10 in the feed configuration, similar to the representation according to FIG. 6. The carrier arms are not shown. FIG. 7 shows a schematic top view of the resectoscope device 10, and FIG. 8 shows a distal view of the resectoscope device 10. The second shaft portion 140 is arranged in the insertion sleeve 12 and is mounted such that it cannot rotate. Said second shaft portion is mounted such that it cannot rotate as a result of the shape of the second shaft portion 140. This portion has an extension 112 (see also FIG. 3), which is larger than the diameter 114. The circle 116 and the diameter 114 are shown again in FIG. 8 for the sake of clarity. For further explanation, reference is made to FIG. 5 and FIG. 4 and the associated description. The extension 112 is arranged in the direction of the major axis 172 and transversely to the longitudinal axis 120 of the shaft 18. The extension 112 is correspondingly larger than the shaft diameter 100. Consequently, the extension is larger than the diameter 114 of the largest circle 116 inscribable in the oval cross-section 110 of the insertion sleeve 12, which, as described, substantially corresponds to the shaft diameter 100. As can be seen in FIG. 8, the second shaft portion 140 is fitted precisely into the insertion sleeve 12. Since the extension 112 substantially corresponds to the length of the major axis 172 (see FIG. 5), rotation of the shaft 18 is blocked. Furthermore, the return (see FIGS. 3 and 4) is closed. Furthermore, a gap of 190 can be seen, through which, for example, the HF resection loop can be guided. The grooves 192 are provided for guiding the carrier arms. Accordingly, the carrier arms, together with the second shaft portion 140 and the HF resection loop, fit into the insertion sleeve 12.

Furthermore, the stereo input optics 160 can be seen. The objectives 162 are circular. Since the end face 41 is arranged obliquely with respect to the longitudinal axis 120 (see FIG. 1), the objectives 162 are shown as oval objectives. In addition, the two LED's 166 can be seen. The objectives 162 are arranged side by side in the direction of the major axis 172 (see FIG. 5), correspondingly in the direction of the greatest extension of the second shaft portion 140. The objectives 162 are designed as lenses and each have a diameter 164. The sum of the diameters 164 of the objectives 162 is larger than the diameter 114 of the largest circle 116 inscribable in the oval cross-section 110 of the insertion sleeve 12 (see FIG. 5). The sum is therefore larger than the shaft diameter 100.

As already described, the feed configuration and the application configuration can be optionally set by the user by the linear displacement of the shaft 18 relative to the insertion sleeve 12 along the longitudinal axis 120 of the shaft 18. If the user wishes to mount the shaft 18 such that, for example, it cannot rotate during a resection procedure, in order to, for example, briefly attend to another task, said user can rotate the shaft 18 about its longitudinal axis 120 in such a way that a major extension axis 168 of the second shaft portion 140, in the direction in which the second shaft portion 140 has its greatest extension, is parallel to the principal axis 172. In this angular position of the shaft 18 relative to the insertion sleeve 12, the second shaft portion 140 can be inserted into the insertion sleeve 12.

1. A resectoscope device, comprising:
an insertion sleeve that can be fed into a cavity of a patient and has an oval cross-section, and
a shaft that extends through the insertion sleeve and has a first shaft portion and a second shaft portion arranged in different positions along a longitudinal axis of the shaft, wherein, at least in an application configuration, the first shaft portion is arranged in the insertion sleeve and is rotatable within the insertion sleeve, and wherein the second shaft portion in the application configuration is arranged outside the insertion sleeve.

2. The resectoscope device as set forth in claim 1, wherein the second shaft portion in at least one direction that is transverse to the longitudinal axis of the shaft has an extension that is greater than the diameter of the largest circle inscribable in the oval cross-section of the insertion sleeve.

3. The resectoscope device as set forth in claim 1, wherein, at least in a feed configuration, the second shaft portion is arranged in the insertion sleeve and mounted such that it cannot rotate.

4. The resectoscope device as set forth in claim 3, wherein the feed configuration and the application configuration can optionally be set by a user by displacing the shaft relative to the insertion sleeve along the longitudinal axis of the shaft.

5. The resectoscope device as set forth in claim 3, wherein a return for a rinsing fluid is formed between the insertion sleeve and the first shaft portion, and
wherein the return is open in the application configuration and closed in the feed configuration.

6. The resectoscope device as set forth in claim 1, wherein the first shaft portion in no direction that is transverse to the longitudinal axis of the shaft has an extension that is greater than the diameter of the largest circle inscribable in the oval cross-section of the insertion sleeve.

7. The resectoscope device as set forth in claim 1, wherein, the application configuration, the first shaft portion in the insertion sleeve is guided linearly along the longitudinal axis of the shaft.

8. The resectoscope device as set forth in claim 1, wherein the first shaft portion and the insertion sleeve collectively form a fit, and
wherein the first shaft portion has an outer contour which at least partially coincides with a circular outer cross-section with a first diameter that is of such a size that the first shaft portion is fitted into the largest circle inscribable in the oval cross-section of the insertion sleeve.

9. The resectoscope device as set forth in claim 1, wherein the first shaft portion is proximal to the second shaft portion.

10. The resectoscope device as set forth in claim 1, wherein the second shaft portion at least partially defines a distal end piece of the shaft.

11. The resectoscope device as set forth in claim 1, wherein the second shaft portion includes an image acquisition apparatus which defines an observation region.

12. The resectoscope device as set forth in claim 11, wherein the image acquisition apparatus includes at least one image acquisition unit which is formed by at least one input optics and/or at least one image sensor for generating images, and
wherein the at least one input optics is arranged on a distal end face of the shaft.

13. The resectoscope device as set forth in claim 12, wherein at least one image acquisition unit is configured for stereo imaging.

14. The resectoscope device as set forth in claim 13, wherein the image acquisition unit has a stereo input optics with two objectives arranged side by side, and wherein the sum of the diameters of the objectives is larger than the diameter of the largest circle inscribable in the oval cross-section of the insertion sleeve.

15. The resectoscope device as set forth in claim 11, wherein the image acquisition device includes at least two image acquisition units directed in different viewing directions.

16. The resectoscope device as set forth in claim 1, further including:
a resection tool,
wherein the shaft and the resection tool can be moved independently of one another relative to the insertion sleeve along the longitudinal axis of the shaft, and
wherein the shaft and the resection tool are designed to be moved within the insertion sleeve after being fed into a cavity during a removal of tissue, wherein the insertion sleeve is in particular designed to rest atraumatically during the removal of tissue.

17. The resectoscope as set forth in claim 16, wherein the resection tool includes two carrier arms and a tool tip, in particular an HF resection loop, arranged between the carrier arms, and
wherein the shaft is arranged in particular between the carrier arms.

18. The resectoscope device as set forth in claim 1, further including a proximal operating module, which is movable as a whole relative to the insertion sleeve.

19. A resectoscope with a resectoscope device as set forth in claim 1.
